(19) United States
(12) Patent Application Publication
(10) Pub. No.: US 2002/0039788 A1
(43) Pub. Date: Apr. 4, 2002

(54) Corneal epithelial graft composites

(76) Inventors: Roslyn R. Isseroff, Sacramento, CA (US); Ivan R. Schwab, Fair Oaks, CA (US)

Correspondence Address: Carol L. Francis, Bozicevic, Field & Francis LLP, Suite 200, 200 Middlefield Road, Menlo Park, CA, 94025, US

(21) Appl. No.: 09/796,995

(22) Filed: Feb. 28, 2001

Related U.S. Application Data
(60) Provisional application No. 60/185,744, filed on Feb. 29, 2000.

(51) Int. Cl. A61K 45/00; C12N 5/08

(52) U.S. Cl. 435/366; 424/93.7

(57) ABSTRACT

The present invention relates to a bioengineered composite graft for the treatment of a damaged or diseased corneal epithelial surface wherein the corneal epithelial composite graft comprises ex vivo corneal epithelial stem cells cultured on an extracellular carrier matrix, the methods of making and using the corneal epithelial composite graft.

### CROSS-REFERENCE TO RELATED APPLICATIONS

This application claims the benefit of U.S. Provisional Application Ser. No. 60/185,744, filed Feb. 29, 2000; which application is incorporated herein by reference in its entirety

### FIELD OF THE INVENTION

The present invention relates to corneal epithelial graft composites and methods of treating damaged corneal surfaces with the corneal epithelial graft composites.

### BACKGROUND OF THE INVENTION

Corneal and conjunctival epithelial cell injury, degenerations and abnormalities are relatively common corneal problems which may threaten one's vision. Ocular surface diseases such as Stevens-Johnson's Syndrome, chemical and thermal burns, ocular surface tumors, immunological conditions, radiation injury, inherited syndromes such as aniridia, and ocular pemphigoid can severely compromise the ocular surface and cause catastrophic visual loss in otherwise potentially healthy eyes. See Tseng, S. C. G., et al., Ophthalmol Clin North Am (1990) 3:595-610. A common pathogenic feature of these seemingly diverse group of diseases is the depletion of the stem cell population from the corneal limbus. Damage or depletion of the corneal stem cells results in the ingrowth of conjunctival elements onto surface of the cornea or “conjunctivization” with associated profound visual loss.

Normally, the corneal surface is composed of 5-6 layers of optically regular epithelium whose homeostasis is maintained by the upward and centripetal migration of epithelial cells derived from stem cells located in the limbus. See Cotsarelis, G., et al., Cell (1989) 57:201-9; and Dua, H.S., et al., STEM CELLS. Ed. C. S. Potten, Academic Press Ltd., London (1977) 331-62. The depletion of the limbal stem cell pool results in an abnormal corneal surface, which cannot be normalized without the reintroduction of a source of stem cells. See Holland, E. J., et al., Trans Am Ophthalmol Soc (1996) 94:677-743; and Tan, D. T., et al., Ophthalmol (1996) 103:29-36. Damage to the corneal and limbal stem cell epithelium can result in significant superficial scarring and degradation of vision despite an otherwise normal eye.

Conventional treatments known in the art are not suitable for diseases, disorders and injuries related to or resulting in loss of stem cells in the corneal limbus. For example, conventional corneal transplantation is not successful in these chronic surface problems since the ultimate success of the therapy is dependent on the gradual replacement of the donor corneal epithelium with the recipient's. See Lindstrom, R. L., et al., N Engl J Med (1986) 315:57-59. This poor prognosis is presumed to be due to the deficiency of limbal stem cells in the recipient eye, which deficiency allows for, or may even stimulate, conjunctival cell ingrowth and the accompanying vascularization and inflammation resulting in corneal graft failure. See Buck, R. C., et al., Curr Eye Res (1986) 5:149-59.

Another conventional treatment involves the repair of damaged corneal surface with amniotic membrane. This approach has been successful in certain ocular surface diseases. See Kim, J. C., et al., Cornea (1995) 14:473-84. Unfortunately, however, this treatment has been unsuccessful in treating surface conditions characterized by stem cell deficiency. See Prabhasawat, P., et al., Arch Ophthalmol (1997) 115:1360-67. In order to correct the stem cell deficiency, these conventional methods combine the amniotic membrane to be transplanted with grafts of limbal tissue, which presumably includes stem cells. These limbal tissue grafts are taken either from the patient's uninvolved eye or from the eye of another donor, which grafts are known as limbal autograft and limbal allograft, respectively. See Kenyon, K. R., et al., Ophthalmol (1989) 96:709-23; Tsubota, K., et al., Ophthalmol (1995) 102:1486-96; and Tsubota, K., et al., N Engl J Med (1998) 340:1697-1703. Unfortunately, this procedure requires harvest of approximately one half or more of the limbus which jeopardizes the donor eye regardless of whether it is autologous or allogeneic. See Coster, D. J., et al., Br J Ophthalmol (1995) 79:977-82; and Jenkins, C., et al., Eye (1993) 7:629-33. Additionally, if the graft is allogeneic, the possible need for long-term immunosuppression presents further problems and complications.

U.S. Pat. Nos. 5,585,265, 5,672,498, and 5,786,201 disclose inventions directed to the production of human corneal epithelial cell strains with extended lifespan. Although the cell strains are derived from human corneal epithelial cells, they are continuous cell strains established by viral infection or plasmid transfection. These cell strains may be useful for in vitro experiments for studying the effects of chemicals and drugs on the human eye, however, these continuous cell strains are inappropriate for human transplantation because of the obvious risk of infection and rejection problems.

Although there have been advances in treating ocular surface diseases, disorders and injuries, which include conjunctival transplants, keratoepithelioplasty, limbal autographs and allografts, there is a long-felt need for a technique which would replace absent stem cells without substantially depleting the stem cell population of the donor limbus. The present invention addresses this need.

### SUMMARY OF THE INVENTION

The present invention relates to a bioengineered composite graft for the treatment of a damaged or diseased corneal epithelial surface wherein the corneal epithelial composite graft comprises ex vivo corneal epithelial stem cells cultured on an extracellular carrier matrix, the methods of making and using the corneal epithelial composite graft.

In one aspect, the invention is directed to a corneal epithelial composite graft comprising an extracellular carrier matrix having a plurality of corneal epithelial cells, which may include a plurality of corneal epithelial stem cells cultured ex vivo upon the extracellular carrier matrix. In one embodiment, the composite graft is a multi-layered epithelium with corneal epithelial differentiation.

In one embodiment of the composite graft, the extracellular carrier matrix is an amniotic membrane, with a human amniotic membrane being of particular interest. In a preferred embodiment, the extracellular carrier matrix comprises collagen. In another embodiment of interest, the extracellular carrier matrix comprises fibrin. In still another embodiment, the extracellular carrier matrix comprises a collagen gel. In another embodiment the collagen (as in a collagen gel) or fibrin (as in a fibrin gel) may further comprise the gelled protein incorporating fibroblasts, derived from the mesenchymal tissue underlying the epithelium being engineered. For example, where the extracellular carrier matrix comprises collagen, as in a collagen gel, the corneal composite graft may be prepared so that corneal stromal fibroblasts are within the gel. Once gelled and contracted, the corneal epithelial cells, comprising corneal epithelial stem cells, are cultivated on this gel. Where the extracellular carrier matrix comprises fibrin, as in a fibrin gel, the fibrin gel may be prepared with corneal stromal fibroblasts incorporated into it, followed by cultivation of corneal epithelial cells, including stem cells, on this surface.

In yet another embodiment, the extracellular carrier matrix comprises a plurality of corneal epithelial cells and a plurality of corneal epithelial stem cells which may be used to treat a damaged or diseased ocular surface.

In another aspect, the invention is directed to a method of making a corneal epithelial composite graft for the treatment of a damaged or diseased ocular surface comprising the steps of preparing an extracellular carrier matrix; isolating a plurality of corneal epithelial stem cells from a donor; and culturing the plurality of corneal epithelial stem cells on the extracellular carrier matrix.

In one embodiment of the method of making a corneal epithelial composite graft according to the invention, the extracellular carrier matrix is an amniotic membrane. In a more preferred embodiment, the extracellular carrier matrix comprises collagen, e.g., as in a collagen gel. The extracellular carrier matrix may be treated with at least one growth factor or at least one attachment factor or a combination of both. Examples of suitable growth and attachment factors include bovine pituitary extract, epidermal growth factor, hepatocyte growth factor, keratinocyte growth factor, hydrocortisone, laminin, tenascin, fibronectin and collagen.

In another embodiment of the method of making a corneal epithelial composite graft according to the invention, the plurality of corneal epithelial cells are obtained from a sample of tissue comprising the plurality of corneal epithelial cells from the superior temporal limbus of a donor eye, washed in a suitable solution or medium, and dissociated to form a single cell suspension. In yet another embodiment, the plurality of corneal epithelial stem cells may be enriched by selective adhesion to an extracellular matrix protein composition such as laminin, tenascin, entactin, hyaluron, fibronectin, fibrin, or collagen, fragments of any such proteins which retain the desired cell-binding activity (e.g., RGD peptides), or a combination thereof.

In another aspect, the invention is directed to a corneal epithelial composite graft for the treatment of a damaged or diseased ocular surface made by preparing an extracellular carrier matrix; isolating a plurality of corneal epithelial stem cells from a donor; and culturing the plurality of stem cells on the extracellular carrier matrix.

In one embodiment of the corneal composite graft, the extracellular carrier matrix is an amniotic membrane. In another embodiment, the extracellular carrier matrix comprises collagen, as in, for example, a collagen gel. The extracellular carrier matrix may be treated with at least one growth factor or at least one attachment factor or a combination of both. Examples of suitable growth and attachment factors include bovine pituitary extract, epidermal growth factor, hepatocyte growth factor, keratinocyte growth factor, hydrocortisone, laminin, tenascin, fibronectin, fibrin, and collagen.

In yet another aspect, the invention is directed to treating a damaged or diseased ocular surface comprising applying a corneal epithelial composite graft to the damaged or diseased ocular surface. In one embodiment of the method of treatment, the composite graft comprises an extracellular carrier matrix having a plurality of corneal epithelial cells including a plurality of corneal epithelial stem cells cultured ex vivo upon the extracellular carrier matrix. In another embodiment, the corneal epithelial composite graft is a multi-layered epithelium with corneal epithelial differentiation. In another embodiment, the extracellular carrier matrix is an amniotic membrane. In an embodiment of particular interest, the amniotic membrane is a human amniotic membrane. In another embodiment, the extracellular carrier matrix comprises collagen, as in, for example, a collagen gel. In another embodiment, the extracellular carrier matrix comprises fibrin, as in, for example, a fibrin gel.

In another aspect, the invention is directed to a method of treating a damaged or diseased ocular surface with a corneal epithelial composite graft made by preparing an extracellular carrier matrix; isolating a plurality of corneal epithelial stem cells from a donor; and culturing the plurality of stem cells on the extracellular carrier matrix. In another embodiment, the extracellular carrier matrix is an amniotic membrane. In one embodiment, the extracellular carrier matrix comprises collagen. In one embodiment, the extracellular carrier matrix comprises fibrin. The extracellular carrier matrix may be treated with at least one growth factor or at least one attachment factor or a combination of both. Examples of suitable growth and attachment factors include bovine pituitary extract, epidermal growth factor, hepatocyte growth factor, keratinocyte growth factor, hydrocortisone, laminin, tenascin, fibronectin, fibrin, and collagen.

In an embodiment of particular interest, the plurality of corneal epithelial cells are obtained from a sample of tissue comprising the plurality of corneal epithelial cells from the superior temporal limbus of a donor eye, washed in a sample in a suitable solution or medium, and dissociated to form a single cell suspension. In yet another embodiment, the plurality of corneal epithelial stem cells may be enriched by selective adhesion to an extracellular matrix protein composition such as laminin, tenascin, entactin, hyaluron, fibronectin, fibrin, or collagen, fragments of any such proteins which retain the desired cell-binding activity (e.g., RGD peptides), or a combination thereof.

In yet another aspect, the invention is directed to a kit comprising the corneal epithelial composite grafts of the invention. The kits may be packaged together with instructions. The kits may include an atmosphere of about 95% O₂/5% CO₂. The kits may also include a transport or incubation chamber wherein the composite graft is sealed until use.

### BRIEF DESCRIPTION OF THE DRAWINGS

FIG. 1 is a histological section of tissue engineered composite cultured corneal graft. Bar=100 μM.

FIG. 2 is a photograph of a preoperative appearance of severe pseudopterygium and upper lid symblepharon (arrowhead) following sustained/alkali burn.

FIG. 3 is a photograph taken of the eye of FIG. 2 three months postoperatively with the sutures removed.

FIG. 4 is a microphotographs of the composite cultured fibrin gel/corneal cells, taken on the days noted.

FIGS. 5 and 6 are photographs of fixed sections of the composite cultured fibrin gel with corneal cells of FIG. 4 taken on days 7 and 13, respectively.

### DETAILED DESCRIPTION OF THE INVENTION

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a cell” includes a plurality of such cells and reference to “the extracellular matrix protein” includes reference to one or more such proteins and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Overview

The present invention relates to bioengineered corneal epithelial grafts suitable for use in repair of ocular surfaces damaged due to, for example, disease, defect, or exposure to a physical or chemical insult. In general, damaged ocular surfaces treated using the grafts of the invention involves loss of a cohort of the tissues' epithelial stem cells. Such the tissues of such damaged surfaces, which are depleted for or otherwise deficient in corneal epithelial stem cells, are generally unable to repair their damages surfaces without replenishing the corneal epithelial stem cell population.

The present invention provides a novel tissue-engineered composite graft containing ex vivo cultured corneal epithelial stem cells which may be used for re-epithelializing a damaged or diseased ocular surface. A suitable extracellular carrier matrix such as amniotic membrane, collagen (e.g., in a collagen gel), fibrin (e.g., fibrin gel), or soft hydrogel contact lenses (e.g., poly-HEMA) can be seeded with cultured corneal epithelial cells, including corneal stem cells, to reconstitute a multi-layered epithelium with comeal epithelial differentiation. By “multi-layered epithelium” is meant that the cells of the graft composite are layered in a manner mimicking the naturally-occurring epithelial layer in vivo. By “epithelial differentiation” is meant that the cells in the graft composite have the phenotypic, histological, or other features of mature epithelial cells of the relevant epithelial tissue in vivo.

Development of non-invasive techniques for cell tagging and tracking allow an analysis of the persistence of the engrafted cells and the topographic geographic mapping of the migration and ultimate localization of donor stem cells. The various laminins known to be present in amniotic membrane can provide signals for hemidesmosomal attachment of epithelium which could help adherence. See Herendael, B. J. et al., Am J Obstet Gynecol (1978) 131:872-880.

This invention assures the transfer of replicative corneal epithelial stem cells, either autologous or allogeneic, to an ocular surface to be treated. The replicative corneal epithelial stem cells allow for continued growth after transplantation and long-term coverage with normal corneal phenotypic cells. Further advantages of the present invention will become hereinafter apparent to one of ordinary skill in the art.

The invention will now be described in more detail.

In one exemplary aspect, the invention features a bioengineered corneal epithelial composite graft comprising ex vivo expanded cultured corneal epithelial stem cells plated onto an extracellular carrier matrix (ECM) which is gradually resorbed in vivo, is at least substantially non-antigenic, facilitates epithelialization without allowing fibrovascular growth, supports epithelial cell differentiation, and contains extracellular matrix components resembling those of the conjunctival basement membrane as described by Prabhasawat, P., et al., Ophthalinol (1997) 104:974-85; Champliud, M. F., et al., J Cell Biol (1996) 132:1189-98; Adinolfi, M., et al., Nature (1982) 295:325-27; and Akle, C. A., et al., Lancet (1981) 11:1003-5 which are herein incorporated by reference. The EMC may comprise basement membrane components such as, for example, laminin, fibronectin, elastin, a variety of integrins, and collagen. A preferred ECM for the composite cultured corneal graft is amniotic membrane. A another preferred ECM is one which comprises collagen, e.g., as in a collagen gel. Still another preferred ECM is fibrin, e.g., as in a fibrin gel. Another ECM of interest is an artificial ECM, such as that provided by a hydrogel, e.g., such as a synthetic hydrogel, e.g., soft hydrogel (e.g., poly-HEMA) matrix. A preferred ECM has characteristics of the original tissue's matrix such that it is flexible, tough, thin, biocompatible, and encourages growth as well as normal cell differentiation. The ECM may also be dissoluble.

In the embodiments of the present invention relating to the treatment of a diseased or damaged ocular surface, it is preferred that the ECM allows the incorporation of corneal keratocytes or corneal stromal fibroblasts within the matrix, or the ECM comprises corneal keratocytes or corneal stromal fibroblasts or both. This allows for “cross talk” between mesenchymal and epithelial cells, or corneal cells or both via secreted cytokines and growth factors. The fibroblasts to be incorporated may be derived from an allogeneic donor eye or from the ipsilateral undamaged or undiseased eye of the patient. Collagen, fibrin, or a similar network-like molecule may form the scaffolding matrix in which the mesenchymal cells are incorporated upon, on or in. The epithelial cells are then be plated on the surface of the ECM matrix and allowed to grow to confluence and become multilayered. If the composite graft is to be used to treat other biological surfaces or membranes, the mesenchymal fibroblast cells found in the tissues of the surface or membrane to be treated would be incorporated into the ECM, and the epithelial cells found in the tissues of the surface or membrane to be treated would be grown on the surface of the ECM matrix.

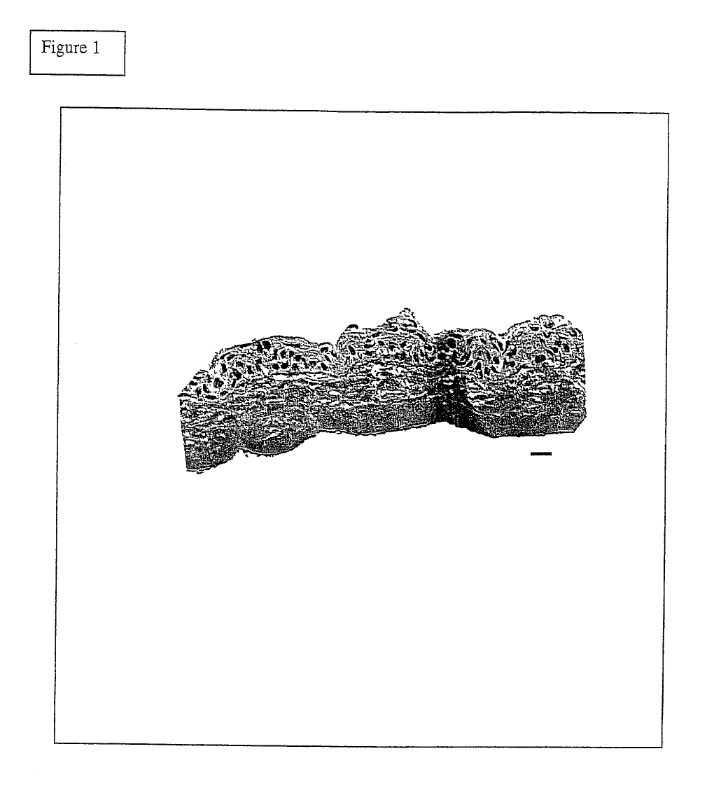

The corneal graft of the present invention demonstrates an architecture which resembles that of a normal cornea, with a multi-layered stratified epithelium as shown in, for example, FIG. 1 and expression of keratin 3 which is a marker for corneal lineage. Human corneal limbal cells were expanded ex vivo and cultivated on a denuded amniotic membrane.

The ECM, such as the amniotic membrane, collagen gel, fibrin gel, hydrogel (e.g., poly-HEMA), or the like, is prepared to enhance the attachment and growth of corneal stem cells. Preparation of amniotic membrane as an ECM is accomplished by the removal of endogenous amniotic epithelial cells by freeze-thawing, enzymatic digestion, and mechanical scraping, followed by treatment of the surface with growth factors, extracellular matrix compounds, and adherence-enhancing molecules. Collagen or fibrin gels can be prepared according to methods well known in the art, and then, just as with the amniotic membrane, treated with growth factors, extracellular matrix compounds, and adherence-enhancing molecules.

The growth factors may include serum, bovine pituitary extract, epidermal growth factor, hydrocortisone and others known in the art. At least one growth factor is used, and such growth factors may be provided in combinations of two or more. The extracellular matrix compounds and adherence-enhancing molecules may include extracellular matrix components such as laminin, tenascin, fibronectin, fibrinogen, fibrin, collagen (e.g., soluble collagen), and others known in the art, as well as naturally occurring, synthetic, or recombinant fragments thereof retaining the desired activity.

Corneal stem cells can be isolated and enriched by harvesting a small biopsy from the normal limbus of the donor eye and separating the epithelium from the stroma by enzymatic digestion so that a single cell suspension is achieved. Then the single cell suspension is allowed to come in brief contact, about 10 minutes, with a surface that has been treated with at least one extracellular matrix component such as laminin, tenascin, entactin, hyaluron, fibronectin, fibrin, or collagen, or naturally occurring, synthetic, or recombinant fragments thereof retaining the desired activity (e.g., RGD peptides). The extracellular matrix components can be combined one with another for combinations of two or more or three or more extracellular matrix components. In one embodiment of particular interest, the extracellular matrix components comprises laminin, tenascin and collagen. The cells that do not adhere are washed away with any suitable sterile solution or medium such as saline or PBS. Then the stem cells are released from the surface by enzymatic digestion and re-plated for expansion in a medium which contains any suitable growth factors. General methods of cell culture can be found in CULTURE OF EPITHELIAL CELLS, 1991 Freshney, R. I. ed., A. John Wiley and Sons, Inc., New York, N.Y., which is herein incorporated by reference.

The cultured cells are then grown and cultured on an ECM surface such as an amniotic membrane surface, a collagen gel, a fibrin gel, or the like by removing the cells from the culture dish with trypsin. Then the cells are re-plated onto the ECM surface that has been pretreated with attachment factors. The culture is either cultivated while submerged or while maintained at the air-liquid interface (e.g., by suspension of the ECM over a stainless steel mesh grid), and growth medium is applied to the underside of the ECM. The culture is propagated and then surgically grafted on the corneal surfaces to be treated. The replicative corneal epithelial stem cells of the present invention allow for continued growth after transplantation and long-term coverage with normal comeal phenotypic cells. These cells also synthesize an extracellular matrix more consistent with that of the native ocular surface and secrete the growth factors and cytokines present in normal epithelium.

The donor eye-derived limbal epithelial cells, which include the population of corneal epithelial stem cells, grew rapidly in the laboratory. Within four weeks, the corneal epithelial cells had expanded sufficiently to produce a bioengineered composite tissue which had an epithelium of about 15-25 mm in diameter with about a 3-5 epithelial cell thickness. Additional corneal epithelial cells may be cryopreserved for future use.

In one example, fourteen patients were treated using corneal graft composites having amniotic membrane as the ECM. Ten patients had autografts and four had sibling allografts wherein the limbal tissue was expanded ex vivo and incorporated into the composite graft which was then transplanted into the eye to be treated. All fourteen reparative procedures were initially successful with no surgical complications.

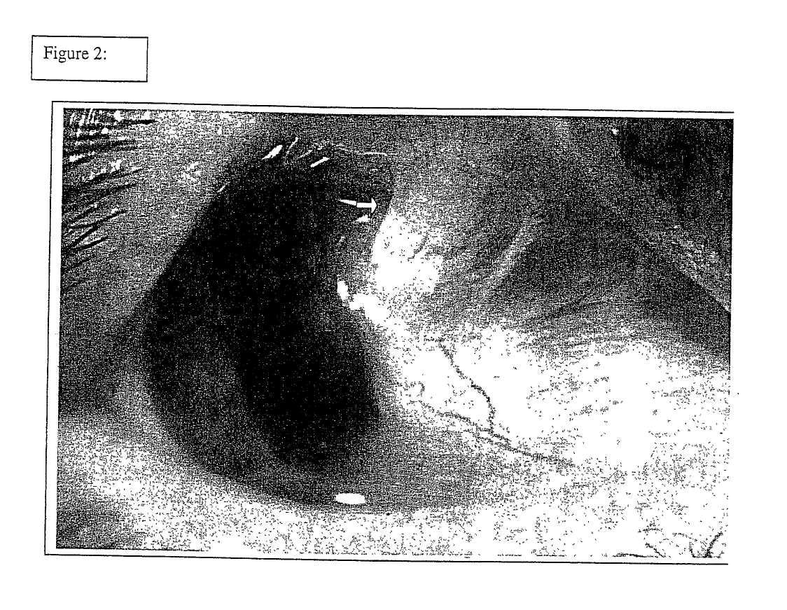

Ten of the fourteen patients had successful results from their treatments with follow-up varying from four to fourteen months, with a median follow-up of eight months. Table 1 (inserted prior to the claims) shows the results of the fourteen patients. FIG. 2 is a photograph of a preoperative appearance of severe pseudopterygium and upper lid symblepharon (arrowhead) following sustained thermal/alkali burn of patient 4. FIG. 3 is a photograph at three months post-operatively with sutures removed of patient four. The edges of resorbing amniotic membrane can be seen. Peripheral corneal vascularization is seen in the mid stroma and could not be removed with superficial dissection. The symblepharon has been relieved.

Success was defined as the complete corneal re-epithelization, stabilized or improved vision, and no recurrence of the corneal disease. Of the ten patients who received autographs, six were defined as successful. Of the four patients who received allografts, all were defined as successful. Patient 3 had bacterial keratitis which delayed his recovery. The patient's surface and systemic immunosuppression was discontinued within the first three weeks. The patient re-epithelialized after two months of treatment with the appropriate topical antibiotics. Patient 6 appeared to lose most of the donor epithelium on the fourth post-operative day. A second cultured allograft, derived from the previously expanded donor cells which had been cryopreserved, was applied and the patient re-epithelialized. This was combined with another procedure, penetrating keratoplasty, and resulted in restoration of vision of 20/80. The patient's immunosuppressive therapy was discontinued within the first month. Patients 13 and 14 had successful transplants. However, complications associated with Cyclosporin A resulted in either the decrease or temporarily discontinuation of the drug.

Complications from the surgical procedures were minimal. There were no complications encountered at the biopsy site of any autologous or allogeneic donor. One patient receiving an autologous composite graft briefly developed a pyrogenic granuloma at the graft/host junction. Complications may arise from a compromised ocular surface, immunosuppression, a therapeutic contact lens, and depletion of the previous barrier through surgery.

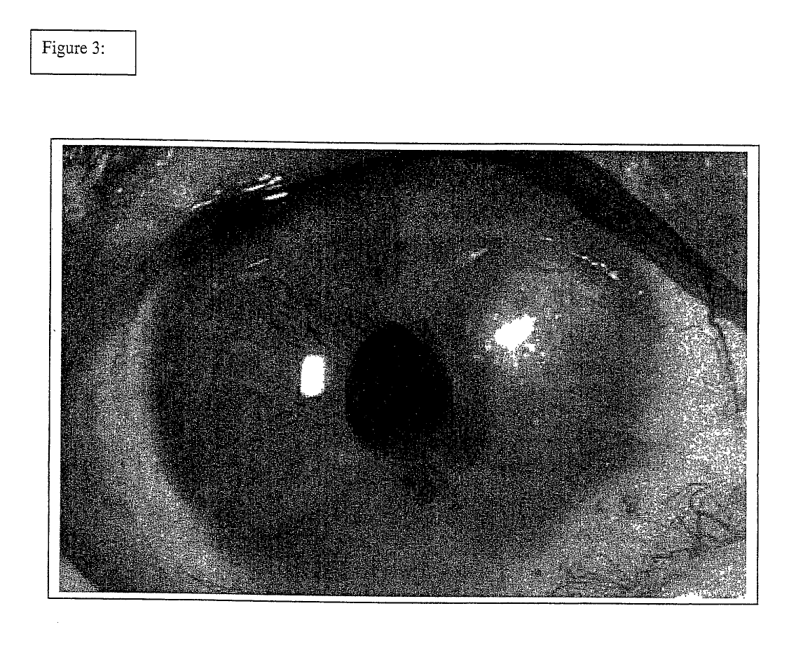

Given the success with corneal graft composites using amniotic membrane, one would reasonably expect that corneal graft composites could be made using similar techniques and substituting other materials for the ECM, e.g., fibronectin, collagen, fibrin, hydrogels, and the like. Indeed, in another specific example, provided herein, corneal graft composites were successfully established using a fibrin gel. FIGS. 4 and 5-6 show photomicrographs of cultured fibrin gel/corneal cells and histology of fixed sections of these cells, respectively.

In general, any subject having a damaged epithelial surface can be treated using composite grafts prepared according to the invention. For example, where the composite graft is a corneal epithelial graft composite, the subject can have any of a variety of corneal and/or conjunctival epithelial cell injuries, degenerations and/or abnormalities, including subjects having ocular surface diseases such as Stevens-Johnson's Syndrome, chemical and thermal burns, ocular surface tumors, immunological conditions, radiation injury, inherited syndromes such as aniridia, ocular pemphigoid, and the like. Of particular interest is the treatment of conditions in which the normal stem cell population of the corneal limbus is depleted, non-functional or otherwise inadequate to promote healing of the corneal damage.

Subjects to be treated include any appropriate subject with mammalian subjects generally being of most interest. Human subjects are of particular interest, however, subjects such as livestock (e.g., horses, cattle, and the like), domesticated animals (e.g., dogs, cats, and the like), and other animals (e.g., those kept in a zoo as breeding animals, particularly for endangered animals) are also of interest.

### EXAMPLES

The following examples are intended to illustrate but not to limit the invention.

### Example 1

### Epithelial Cell Harvest and Corneal Stem Cell Growth

A referred sample of seven patients with limbal stem cell deficiency and seven patients with other sever ocular surface disease that had not been managed successfully with currently available techniques were selected for treatment (Table 1; inserted prior to the claims). Institutional Review Board approval was sought and secured for this procedure; informed consent was obtained from patients and donors, and all human subjects treated according to the Helsinki Accord.

Corneal epithelial cell harvest was performed in a similar manner whether cells were autologous or allogeneic. A 2 mm² biopsy was obtained from the superior temporal limbus to include the limbal conjunctiva up to and including the peripheral corneal epithelium and was placed aseptically in a petri dish. The biopsy was washed in phosphate buffered saline (PBS) and then minced and dissociated by incubation in trypsin/EDTA or trypsin and dispase. Optionally, the procedure can involve a panning step to enrich for stem cells in the population. Panning is accomplished by placing a single cell suspension from the biopsy on a plastic culture dish coated with a combination of extracellular matrix proteins (referred to herein as an “extracellular matrix protein composition”) including laminin, collagen and tenascin to which corneal stem cells selectively adhere for a short time of about 5 to about 15 minutes. This step is not required, and was not used in producing grafts for use in the human patients as described below.

The tissue was incubated in a solution of trypsin/EDTA solution for 30 minutes at 37° C. in a 5% CO₂ incubator. The action of the trypsin was inhibited by adding an equal volume of medium that contained 10% fetal bovine serum. The samples was minced with a scalpel, and tissue aggregates removed by centrifugation at about 800×g for about 5-7 minutes.

The single cell suspension was ex vivo expanded and cultivated using a modification of the method originally described by Rheinwald, J. C., et al., Cell (1975) 6:331-43, which is incorporated herein by reference. In this technique, the cells are initially expanded by growth on a feeder layer of replication defective, but metabolically active fibroblasts (3T3 cells). More specifically, the cells were plated at about 1.0×10⁶ cells/ml GM (Growth Medium: Dulbecco's Modified Eagle's Medium, fetal calf serum, glutamine, ABAM, epidermal growth factor, hydrocortisone, and cholera toxin) on 2,100 mm dishes with mitomycin C-treated 3T3 cells. The 3T3 cells have been treated and trypsinized. The dishes containing the corneal cells and the 3T3 feeder cells were placed into a 37° C./5% CO₂ incubator. Within about 3 days small colonies formed. Once the epithelial colonies are established (reach 40-50% confluency), the feeder cells were removed, and the corneal epithelial cells are further expanded by growth in a serum-free, low calcium medium such as Keratinocyte Growth Medium, KGM (Cascade Biologics, Portland, Oreg.).

Optionally, the cells can be cultivated without feeder cells such as 3T3 cells, in which case the cells are cultivated in serum-free medium with a growth factor and/or attachment factor such as bovine pituitary extract.

Cultures in logarithmic growth phase were trypsinized and replated onto a extracellular carrier matrix as described below. Some cells were cryopreserved for later use. Cells suspended in 50% Keratinocyte Growth Medium/40% fetal calf serum/10% dimethyl sulfoxide were cryopreserved in liquid nitrogen using a freezing rate of −1° C./min in a CryoMed biologic freezer.

### Example 2

### Preparation of Human Amniotic Membrane and Growth of Corneal Cells

Human amniotic membrane (HAM), prepared for human use and obtained from Bio-Tissue (Miami, Florida), was shipped frozen and stored at −80 C.

Immediately prior to use, the HAM was rapidly thawed in a 37 C water bath, washed with PBS or saline and the amniotic epithelium was removed by using a combination of trypsin digestion and mechanical scraping. Complete removal of the amniotic epithelial cells was conducted and confirmed by microscopy by a method such as that described below. The HAM, epithelial side up, was draped over a circular sterile stainless steel mesh support with a 1.5×1.5 cm square cut in its center. Optionally, the surface of the membrane can be treated with a combination of growth factors and attachment factors to enhance the attachment of the epithelial cells.

Cultures of corneal cells in the logarithmic growth phase (prepared according to Example 1) were trypsinized and re-plated onto the surface of the previously prepared, substantially acellular human amniotic membrane at a density of about 1.5-3×10⁶ cells/2 mm². The presence of stem cells in the limbal cultures was confirmed by assessing the colony forming efficiency of representative cultures. Only stem cells can establish colonies of greater than 50 cells from an individual founder cell. The number of founder stem cells in representative limbal cell cultures varied from about 2% to about 9% of the total cell population as determined by techniques described by Rheinwald, J. C. et al. The cells were cultivated on the membrane in an atmosphere of about 5% CO₂ at 37 C for an additional 10 to 14 days, until a multi-layered epithelium was obtained.

Immediately before grafting, the tissue culture medium was aspirated and the composite cultured tissue was washed extensively with PBS or saline before transfer into fresh un-supplemented medium. The tissue was placed in a sealed transport/incubation chamber which had been purged with about 95% O₂/5% CO₂ and transported to the operating room. Stains including H & E, and immunocytochemical stains of AE1 (ICN Biomedicals, Inc, Aurora, Ohio) were used for staining the composite graft to confirm epithelial cell growth and adherence. A representative section of the amniotic membrane with the attached multi-layered corneal epithelial cells is shown in FIG. 1.

### Example 3

### Surgical Technique

A similar surgical repair technique was used, with slight modification depending on the disease process, for each patient. All patients had the abnormal ocular surface tissue removed from the bulbar and, if present, the palpebral, the conjunctival surfaces and the corneal surface (see Table 1, inserted prior to the claims). Full thickness penetrating or lamellar keratoplasty was performed. The posterior peripheral edge of the amniotic membrane was sewn into the peripheral recessed/resected conjunctiva and anteriorly to the cornea with 10-0 nylon, if the cornea was not covered completely, and a planotherapeutic bandage contact lens was placed to prevent lid trauma. The contact lens remained in place for a period of about 2-3 months, if possible.

Patients were reevaluated on a biweekly basis, and during this time, the amniotic membrane was noted to gradually resorb. The epithelium appeared to attach, allowing for removal of the peripheral conjunctival sutures. Cyclosporin therapy, both oral, about 200 mg daily, and topical, about 2%, was initiated immediately post operatively in patient who received the allografts.

Patients treated using the human corneal epithelial composite grafts, and the results obtained with such grafts, are described in Table 1 (inserted prior to the claims).

### Example 4

### Preparation of Human Amniotic Epithelium from Placentas

Fresh amniotic membrane was secured from two fresh placentas from the University of California, Davis Medical Center three to four days following delivery of a healthy infant. Institutional Review Board approval was obtained for the harvest of these placental tissues, and informed consent was obtained from each postpartum mother shortly after birth. Each mother who donated the amniotic membrane had been tested and cleared for HIV 1 and 2, hepatitis C, and syphilis even though this protocol was only for investigational purposes. Prior to removal of the amniotic membrane, the placentas had been kept at 4 C for three to four days to be certain that the infant was healthy and no further pathological examination was required of the placenta.

The amniotic membrane was harvested in the following manner. The amnion was dissected from the placenta in a sterile hood with blunt dissection only. Once the amnion had been dissected from the placenta, it was cleaned, and rinsed in normal saline. The amniotic membrane was cut into squares approximately 40 mm² and placed in storage medium. The membrane was then rapidly frozen to −80° C. As amniotic membrane was needed, it was individually thawed.

A variety of combinations of trypsinization, sonification, scraping and washing were studied until the simplest, most effective method of complete removal of amniotic epithelium with preservation of the histological appearance of the basement membrane of the amnion was found. It was determined that the simplest and most effective method of removal of the amniotic membrane and preservation of the HAM basement membrane was trypsinization for 15 minutes followed by gentle scraping with a pair of blunt forceps. Adherence of the expanded epithelial cells from presumed limbal corneal epithelial stem cells was successful.

These techniques included sonification for 15, 30, 45, or 90 minutes followed by gentle scraping of the epithelial surface, or sonification for 15, 30, 45, or 90 minutes with trypsinization for 15 minutes, followed by gentle scraping of the epithelial surface or trypsinization for 15 minutes, followed by gentle scraping of the epithelial surface followed by 15, 30, 45, and 90 minutes of sonification. Additionally, trypsinization for 15, 30, 60, 90, and 120 minutes followed by gentle scraping was performed.

The method of trypsinization and scraping is as follows: 3 ml of Dulbecco's Phosphate Buffered Saline (DPBS, Gibco/BRL Life Technologies, Gaithersburg, Md.) with 1% antibiotic-antimycotic(ABAM): 10,000 u Penicillin-G/ml and 10,000 mcg/mL Streptomycin with 25 mcg/ml fungizone (Gemini Bio-Products, Inc., Calabasas, Calif.) (PBS/1% ABAM) was placed in 60 mm tissue culture dish. Into each dish was placed a 1×1 inch amniotic membrane using forceps. The PBS was aspirated gently taking care not to take the amniotic membrane. The membrane was washed twice more with PBS/1% ABAM or the membrane may be washed with a similar antibiotic-antifungal solution. Then, three ml of 0.25% trypsin/0.01 mM EDTA was placed in the culture dish. The dish was placed in an incubator at 37° C. for the specified time as described above. Control amniotic membrane was placed through the same procedure with was covered with PBS. After the chosen time, the trypsin was neutralized with 3 ml Dulbecco's Modified Eagles Medium (Gibco/BRL Life Technologies, Gaithersburg, Md.) with 10% Fetal Calf Serum (Gemini Bio-Products, Inc., Calabasas, Calif.). The amniotic membrane was again rinsed with PBS/1% ABAM. The epithelial layer was scraped off using blunt forceps. The membrane was then washed twice with PBS/1% ABAM, and fixed with Streck Tissue Fixative.

These portions of amniotic membrane were fixed and stained with H & E (hematoxylin and eosin stain) and reviewed in a masked fashion by two observers with agreement between the two observers. In each of these combinations the presence or absence of amniotic membrane epithelium, and the quality of the underlying basement membrane was assessed by histology to determine the best method of removal and preservation of the basement membrane of the amniotic membrane. Comparison was made to the normal non-trypsinized amniotic membrane.

### Example 5

### Cultivation of Corneal Epithelial Cells on Fibrin Gel Substrates

Corneal fibroblasts were cultivated from human corneas by explant culture. The corneal stroma, devoid of epithelium (removed by Dispase) was minced to pieces approximately 1 mm², and the pieces allowed to adhere to the surface of a tissue culture dish by air drying for 30 minutes. After the tissue was adhered, culture medium (Dulbecco's MEM with 10% fetal calf serum (DMEM), 10% fetal calf serum (FCS)) was added, and changed every 3 days. At day 7 fibroblasts were seen migrating from the explants onto the tissue culture dish surface. At day 14 the fragments of tissue are removed, and the fibroblasts released from the plate by trypsinization and recovered by centrifugation. They were cryopreserved and recovered as needed, by seeding in DMEM 10% FCS.

The corneal epithelial cells were then cultured to second passage as described above in Example 1 above.

A fibrin gel was cast in the wells of a 24 well plate using the following protocol: One ml of a 7 mg/ml fibrinogen solution (plasminogen-free fibrinogen, human, Calbiochem, San Diego, Calif.) in distilled water is combined with 250 μl of 50 nM CaCl2, 50 μl of 2.5 mg/ml aprotonin (Sigma) in Tris Buffer pH7.2 at room temperature. A suspension of previously cultured corneal fibroblasts were added, 100 μl containing 7.5×10⁵ cells. When these components were thoroughly mixed, 500 μl of thrombin, 20 U/ml (Sigma) in Tris Buffer pH7.2, was added, quickly vortexed to mix and 300 μl of the resulting fibrinogen solution pipetted into each well. The final fibrinogen suspension contained 3.5 mg fibrinogen, 2.5 mM Ca⁺⁺, and 2 NIH units thrombin. The plate was placed at 37° C. for 60 minutes for polymerization of the fibrin gel.

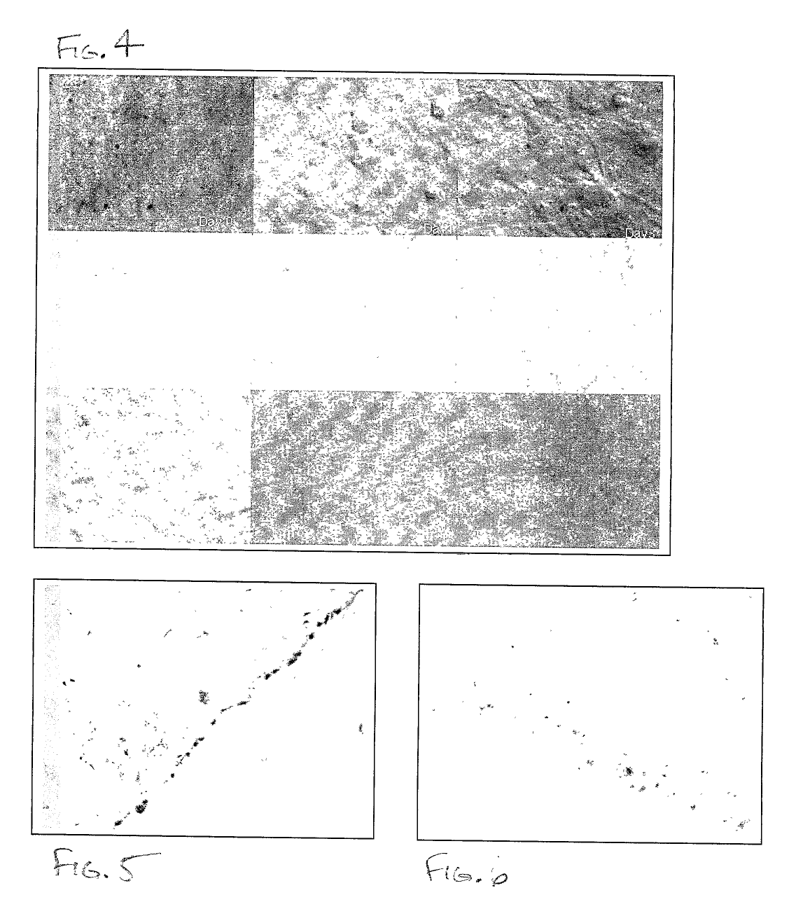

One ml of Cornea Growth Medium (CGM) (Epilife, containing Human Corneal Growth Supplement, both from Cascade Biologics, Inc.) was added to each well after polymerization. Twenty four hours later the corneal epithelial cells were trypsinized from their culture dish, resuspended in CGM medium supplemented with 20 μg/ml aprotonin and seeded on the prepared fibrin gels, 4×10⁶ cells/1 ml/well. Medium was changed every other day. Cell growth is monitored using inverted microscopy (FIG. 4) and with histology of fixed sections taken on days 7 (FIG. 5) and day 13 (FIG. 6).

This example shows that other materials can be substituted for the amniotic membrane in the production of corneal graft composites.

The foregoing description has been presented for purposes of illustration and description. It is not intended to be exhaustive or to limit the invention. The accompanying drawings are included to provide a further understanding of the invention and are incorporated in and constitute a part of this specification, illustrate several embodiments of the invention and together with the description serve to explain the principles of the invention. Obvious modifications or variations are possible in light of the above teachings. All such obvious modification and variations are intended to be within the scope of the present invention.

All publications, patents, and patent applications mentioned above are incorporated by reference herein to the extent they supplement, explain, provide background information, or teach methodology, techniques or compositions employed herein.

TABLE 1
Composite Epithelial Grafts for Ocular Surface Diseases

| Patient | | | | | | |
|---|---|---|---|---|---|---|
| Case | | | | | | |
| Number | Type of Graft | | | | Length of | |
| Age | Source of Cells | | Pre Op | Post Op | Followup | |
| Sex | Carrier | Indication | Vision | Vision | Months | Result |
| VH | Autologous | Bulbar and palpebral CIN | 20/200 | 20/100 | 14 | Smooth surface |
| 1 | Contralateral eye | recurrence involving 270° of limbus | | | | No recurrence |
| 52 | Amniotic membrane | | | | | Complete re- |
| Male | | | | | | epithelialization |
| | | | | | | Successful |
| VO | Autologous | Recurrent pteryglum | 20/20 | 20/20 | 14 | Recurrence of ptergium; |
| 2 | Ipsilateral eye | Two previous surgical excisions | | | | No restriction |
| 42 | Amniotic membrane | Motility restriction | | | | Unsuccessful |
| Female | | | | | | |
| JB | Allograft | Bilateral pseudopemphigoid and | Count | 20/60 | 14 | Successful epithelial |
| 3 | Sibling (female) | stem cell failure from chronic | fingers | | | resurfacing with |
| 73 | Amniotic membrane | medication administration with | | | | vascularization |
| Male | | conjunctivalization of cornea | | | | Infectious crystalline |
| | | | | | | keratopathy delayed |
| | | | | | | healing |
| | | | | | | Healed epithelium |
| | | | | | | eventually |
| | | | | | | Successful |
| JR | Autologous | *Severe thermal/chemical burn | Count | 20/30 | 13 | No recurrence of corneal |
| 4 | Contralateral eye | Stem cell destruction | fingers | | | disease |
| 40 | Amniotic membrane | Conjunctivization of surface | | | | Completely re-epithelial- |
| Male | | | | | | ized |
| | | | | | | Successful |
| RM | Autologous | Pseudopterygium with motility | 20/25 | 20/25 | 12 | Completely re-epithelial- |
| 5 | Contralateral eye | Restriction (scarring with | | | | ized |
| 33 | Amniotic membrane | vascularization secondary to | | | | Clear cornea |
| Male | | unknown trauma resembling | | | | Recurrence at 7 months |
| | | pterygium temporally) | | | | Unsuccessful |
| LB | Allograft | *Severe alkali burn 1986, dense | Hand | 20/100 | 12 | Lost contact lens |
| 6 | Sibling (female) | superficial vascularization OU | motion | | | Lost epithelium with |
| 46 | Amniotic membrane | | | | | reapplication of cells |
| Male | | | | | | with collagen shield and |
| | | | | | | contact lens |
| | | | | | | Re-epithehialized cornea |
| | Secondary penetrating | | | | 6 months | Secondary PKP |
| | keratoplasty | | | | following | Successful |
| | | | | | secondary | |
| | | | | | PKP | |
| MJ | Autologous | Inferior pseudopterygia 180° of | 20/20 | 20/20 | 12 | Successfully re- |
| 7 | Ipsilateral eye | cornea - vascularization and fibrous | | | | epithelialized |
| 46 | Amniotic membrane | scarring at interior 180° of cornea 4- | | | | Clear cornea, no |
| Male | | 5 mm onto cornea circumferentially- | | | | vascularization |
| | | Unknown cause | | | | Successful |
| FV | Autologous | Recurrent presumed pterygium with | 20/20 | 20/25 | 10 | No recurrence |
| 8 | Contralateral eye | motility restriction | | | | Complete re-epitheliali- |
| 49 | Amniotic membrane | | | | | zation of cornea |
| Male | | | | | | No restriction |
| | | | | | | Successful |
| FM | Aulologous | *Severe alkali burn | Hand | 20/100 | 5 | Initial re-epithelialization |
| 9 | Contralateral eye | Non-healing defect | motion | (Cataract) | | Epithelial loss |
| 48 | Amniotic membrane | Corneal melt | | | | Chronic epithelial defect |
| Male | Simultaneous pene- | Corneal perforation | | | | Unsuccessful |
| | trating keratoplasty | | | | | |
| PS | Autologous | Severe recurrent ptergyium | 20/25 | 20/25 | 5 | Re-epithelialization |
| 10 | Contralateral eye | Two recurrences and S/P radiation | | | | No recurrence |
| 41 | Amniotic membrane | | | | | Successful |
| Male | Simultaneous lamellar | | | | | |
| | graft | | | | | |
| KR | Autologous | Severe alkali burn unilateral | Hand | CF | 5 | Complete re-epitheliali- |
| 11 | Contralateral eye | | motion | | | zation |
| 34 | Amniotic membrane | | | | | Conjunctivalization with |
| Male | Corneal transplant | | | | | vascularization |
| | | | | | | Unsuccessful |
| GF | Autologous | Recurrent pterygium ×3 | 20/20 | 20/25 | 6 | No recurrence |
| 12 | Contralateral eye | Pterygium increased 4 mm onto the | | | | Completely re-epithelial- |
| 39 | Amniotic membrane | cornea toward the visual axis | | | | ization |
| Female | | | | | | Successful |
| LF | Allogeneic | Stevens-Johnson syndrome | Count | 20/200 | 3 | Re-epithelialization |
| 13 | Daughter | | fingers | | | Clear graft |
| 81 | Amniotic membrane | | | | | Had to decrease |
| Female | Corneal transplant | | | | | Cyclosporin |
| | | | | | | Successful |
| GN | Allogeneic | *Severe alkali burn | Hand | 20/200 | 3 | Re-epithelialization |
| 14 | Son | | motion | | | Clear graft |
| 68 | Amniotic membrane | | | | | Had to temporarily |
| Male | Corneal transplant | | | | | discontinue Cyclosporin |
| | | | | | | Successful |

*Pfister classification
CIN, corneal intraepithelial neoplasia; HM, hand motion vision.; S/P, status post; PKP, penetrating keratoplasty.

What is claimed is:

1. An isolated corneal epithelial composite graft comprising:
an extracellular carrier matrix; and
a plurality of corneal stem cells, which corneal stem cells are associated with the extracellular carrier matrix by ex vivo culturing of the corneal stem cells upon said extracellular carrier matrix.

2. The isolated corneal epithelial composite graft of claim 1, wherein said composite graft comprises a multi-layered epithelium comprising differentiated corneal epithelial cells.

3. The isolated corneal epithelial composite graft of claim 1, wherein the extracellular carrier matrix is an amniotic membrane.

4. The isolated composite graft of claim 1, wherein the extracellular carrier matrix comprises collagen.

5. The isolated composite graft of claim 1, wherein the extracellular carrier matrix comprises fibrin.

6. An isolated corneal epithelial composite graft comprising:
an extracellular carrier matrix; and
a multi-layered corneal epithelium comprising a plurality of differentiated corneal cells, the corneal cells being associated with the extracellular carrier matrix by ex vivo culturing of corneal stem cells upon said extracellular carrier matrix.

7. The isolated corneal epithelial composite graft of claim 6, wherein the extracellular carrier matrix comprises a material selected from the group consisting of an amniotic membrane, collagen, and fibrin.

8. A method of making a corneal epithelial composite graft, the method comprising
preparing an extracellular carrier matrix;
isolating a plurality of corneal epithelial stem cells from a donor; and
culturing ex vivo the plurality of corneal epithelial stem cells on the extracellular carrier matrix;
wherein an corneal epithelial composite graft comprising epithelial stem cells is produced.

9. The method of claim 8, wherein the corneal epithelial composite graft is adapted for treatment of a damaged or diseased ocular surface.

10. The method of claim 8, wherein the extracellular carrier matrix comprises an amniotic membrane.

11. The method of claim 8, wherein the extracellular carrier matrix comprises collagen.

12. The method of claim 8, wherein the extracellular carrier matrix comprises fibrin.

13. The method of claim 8, wherein the step of preparing the extracellular carrier matrix further comprises:
treating the extracellular matrix with at least one growth factor or at least one attachment factor, wherein said growth factor or said attachment factor is selected from the group consisting of: bovine pituitary extract, epidermal growth factor, hepatocyte growth factor, keratinocyte growth factor, hydrocortisone, laminin, tenascin, fibronectin or collagen.

14. The method of claim 8, further comprising the step of enriching the plurality of stem cells with an extracellular matrix protein composition.

15. The method of claim 14, wherein the extracellular matrix protein composition comprises laminin, collagen, tenascin or a combination thereof.

16. The method of claim 8, wherein the epithelial stem cells are corneal epithelial stem cells, and wherein the step of isolating the plurality of stem cells further comprises:
obtaining a sample of tissue comprising the plurality of stem cells from the superior temporal limbus of the eye of the donor;
washing the sample in a suitable solution or medium; and
dissociating the plurality of stem cells to form a single cell suspension.

17. The method of claim 16, further comprising the step of:
adhering the plurality of stem cells in the single cell suspension to a surface coated with an extracellular matrix protein composition, wherein the extracellular matrix protein composition comprises laminin, collagen, tenascin, or a combination thereof.

18. The method of claim 8, wherein the plurality of stem cells are cultured on the extracellular carrier matrix until a multiple cell layer is obtained.

19. The method of claim 18, wherein the multiple cell layer comprises a multi-layered epithelium.

20. A method of making a corneal epithelial composite graft, the method comprising
preparing an extracellular carrier matrix;
isolating a plurality of corneal epithelial stem cells from a donor; and
culturing ex vivo the plurality of corneal epithelial stem cells on the extracellular carrier matrix;
wherein a corneal epithelial composite graft comprising corneal epithelial stem cells is produced.

21. The method of claim 20, wherein the extracellular carrier matrix comprises a material selected from the group consisting of an amniotic membrane, collagen, and fibrin.

22. The method of claim 20, wherein the step of preparing the extracellular carrier matrix further comprises:
treating the extracellular carrier matrix with at least one growth factor or at least one attachment factor, wherein said growth factor or said attachment factor is selected from the group consisting of: bovine pituitary extract, epidermal growth factor, hepatocyte growth factor, keratinocyte growth factor, hydrocortisone, laminin, tenascin, fibronectin or collagen.

23. The method of claim 20, further comprising the step of:
enriching the plurality of stem cells for stem cell by contacting the cells to a surface coated with an extracellular matrix protein composition, wherein the extracellular matrix protein composition comprises laminin, collagen, tenascin, or a combination thereof.

24. The method of claim 20, wherein the step of isolating the plurality of corneal epithelial stem cells further comprises:
obtaining a sample of tissue comprising the plurality of stem cells from the superior temporal limbus of the eye of the donor;
washing the sample in a suitable solution or medium;
dissociating the plurality of stem cells to form a single cell suspension; and
adhering the plurality of stem cells in the single cell suspension to a surface coated with an extracellular matrix protein composition, wherein the extracellular matrix protein composition comprises laminin, collagen, tenascin, or a combination thereof.

25. The method of claim 20, wherein the plurality of stem cells are cultured on the extracellular carrier matrix until a multiple cell layer is obtained.

26. An isolated composite graft for the treatment of a damaged or diseased biological surface made according to the method of claims 8 or 20.

27. An isolated comeal epithelial composite graft comprising:
an extracellular carrier matrix comprising an amniotic membrane; and
a plurality of corneal stem cells, which corneal stem cells are associated with the extracellular carrier matrix by ex vivo culturing of the corneal stem cells upon said extracellular carrier matrix.

28. The isolated corneal epithelial composite graft of claim 27, wherein said composite graft comprises a multi-layered epithelium comprising differentiated corneal epithelial cells.

29. The isolated corneal epithelial composite graft of claim 27, wherein the extracellular carrier matrix is a human amniotic membrane.

30. The isolated corneal epithelial composite graft of claim 27, wherein the corneal epithelial cells are human corneal epithelial cells.

31. An isolated corneal epithelial composite graft comprising:
an extracellular carrier matrix comprising an amniotic membrane; and
a multi-layered corneal epithelium comprising a plurality of differentiated corneal cells, the corneal cells being associated with the extracellular carrier matrix by ex vivo culturing of corneal stem cells upon said extracellular carrier matrix.

32. An isolated corneal epithelial composite graft comprising:
an extracellular carrier matrix comprising fibrin; and
a plurality of corneal stem cells, which corneal stem cells are associated with the extracellular carrier matrix by ex vivo culturing of the corneal stem cells upon said extracellular carrier matrix.

33. The isolated corneal epithelial composite graft of claim 32, wherein said composite graft comprises a multi-layered epithelium comprising differentiated corneal epithelial cells.

34. The isolated corneal epithelial composite graft of claim 32, wherein the corneal epithelial cells are human corneal epithelial cells.

35. An isolated corneal epithelial composite graft comprising:
an extracellular carrier matrix comprising fibrin; and
a multi-layered corneal epithelium comprising a plurality of differentiated corneal cells, the corneal cells being associated with the extracellular carrier matrix by ex vivo culturing of corneal stem cells upon said extracellular carrier matrix.

36. A method of treating a damaged or diseased corneal epithelial surface, the method comprising applying the corneal epithelial composite graft of claims 1, 6, 27, 31, 32, or 35 to a damaged or diseased corneal epithelial surface of a subject.

37. A method of treating a damaged or diseased biological surface comprising applying a corneal epithelial composite graft made according to the method of claims 8 or 22 to a damaged or diseased epithelial surface of a subject.

38. A kit for treating a damaged or diseased biological surface comprising the composite graft according to claims 1, 6, 27, 31, 32, or 35.

39. A kit for treating a damaged or diseased biological surface comprising a composite graft made according to the method of claims 8 or 22.
